(19) United States
(12) Patent Application Publication
(10) Pub. No.: US 2013/0259929 A1
(43) Pub. Date: Oct. 3, 2013

(54) USE OF A PHOSPHOPEPTIDE ABLE TO BLOCK HER3/P85 INTERACTION FOR THE TREATMENT OF HER2 HYPER-EXPRESSING TUMOURS

(71) Applicants: Rita Falcioni, Roma (IT); Valentina Folgiero, Roma (IT); Selene Eleonora Di Carlo, Torvaianica (RM) (IT); Giulia Bon, Roma (IT)

(72) Inventors: Rita Falcioni, Roma (IT); Valentina Folgiero, Roma (IT); Selene Eleonora Di Carlo, Torvaianica (RM) (IT); Giulia Bon, Roma (IT)

(21) Appl. No.: 13/877,450

(22) PCT Filed: Sep. 29, 2011
(86) PCT No.: PCT/IT2011/000336

§ 371 Date: Jun. 19, 2013

(30) Foreign Application Priority Data
Oct. 4, 2010 (IT) RM2010A000517

(51) Int. Cl. A61K 38/10; A61K 45/06; A61K 39/395; C07K 7/08

(52) U.S. Cl. CPC A61K 38/10; C07K 7/08; A61K 45/06; A61K 39/39558
USPC 424/450; 514/19.2; 536/23.5; 424/133.1; 514/19.8; 514/44 R; 424/178.1; 435/320.1; 530/326

(57) ABSTRACT

The present invention concerns the use of a phosphopeptide able to block HER3/p85 interaction for the treatment of HER2 hyper-expressing tumours, such as, for example, metastatic mammary tumour, possibly in combination with other anti-tumour agents like, for example, trastuzumab

The present invention concerns the use of a phosphopeptide able to block HER3/p85 interaction for the treatment of HER2 hyper-expressing tumours. Particularly, the invention concerns the use of a phosphopeptide suitable to block HER3/p85 interaction for the treatment of HER2 hyper-expressing tumours such as, for example, metastatic mammary tumour, possibly in combination with other anti-tumour agents like, for example, trastuzumab.

The family of epidermal growth factor receptors (EGFR)/ErbB comprises four members: EGFR (also known as ErbB1/HER1), ErbB2 (Her2/Neu), ErbB3 (HER3) ed ErbB4 (HER4). Said receptors, having tyrosine-kinase activity, play a fundamental role in cell survival, proliferation and differentiation, in addition to neo-plastic transformation. These functions are mediated by a complex network of intracytoplasmic signal transduction pathways triggered through interaction of receptor with relevant ligand. This interaction results in heterodimerization of receptors, phosphorylation of cytoplasmic domains thereof and consequent activation of kinase domain required in order above said processes to be triggered. Heterodimerization is preferred over homodimerization and among said receptors HER2 is preferred heterodimerization partner by other family members. Therefore, activation and consequently function thereof in normal tissues is essentially dependent on heterodimerization partner (1). In various epithelial origin tumours HER2 receptor is often hyper-expressed and has been showed that hyper-expression thereof in normal cells induces neoplastic transformation. Under these conditions the receptor is homodimerized becoming constitutively active, that is it increases phosphorylation levels of MAPK kinase resulting in hyiper-proliferation and for this reason it has been defined oncogene (2-3).

The amplification of HER2 oncogene occurs in approximately 25% of mammary tumours and is associated with unfavourable prognosis (4). Therapy with Trastuzumab (Herceptin), humanized monoclonal antibody against HER2 receptor extracellular domain, results in favourable clinical response for oncogene hyper-expressing primary mammary tumours. The binding of said antibody to HER2 receptor results in homodimer dissociation with subsequent internalization of receptor that is degraded and thus receptor-induced proliferative signal is disrupted (5). On the contrary, in metastatic mammary carcinomas the Hercetin therapy controls the disease such that within 12 months there is a disease progression. The causes of pharmacological resistance development to the treatment with Herceptin antibody have been referred to various molecular o mechanisms, but surely a most accepted one is the activation of PI3K kinase survival signal (6-8). Normally in nature, highest PI3K activator is HER-2/HER-3 heterodimer since HER3 has six binding domains to p85, i.e. PI3K activity regulating sub-unit (9). Often HER2 hyper-expressing metastatic mammary tumours have high levels of HER3 expression (10-11), characteristics allowing HER2/HER3 heterodimer to induce high activation of PI3K and therefore to contrast the effects of Hercetin treatment.

In the light of above it is therefore apparent the need to provide for new compounds or molecules suitable to contrast the proliferation and cellular survival and favour the response to Herceptin therapy, whereas the latter is ineffective.

During the last ten years various drugs suitable to inhibit, at various levels, PI3K kinase signal pathway have been developed. Many of these drugs actually are at clinical trial step, but up to now none is clinically used for treatment of HER2 oncogene hyper-expressing mammary metastatic tumours (12).

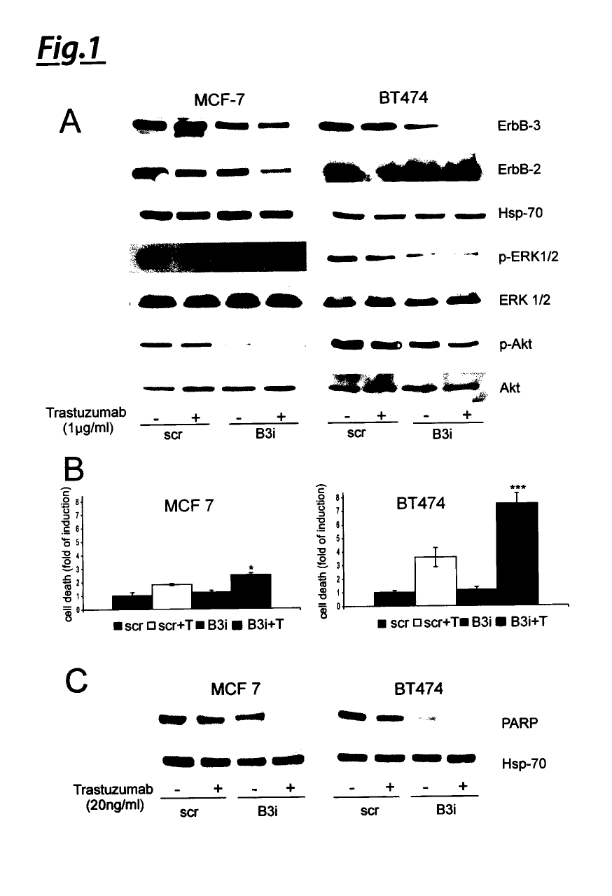

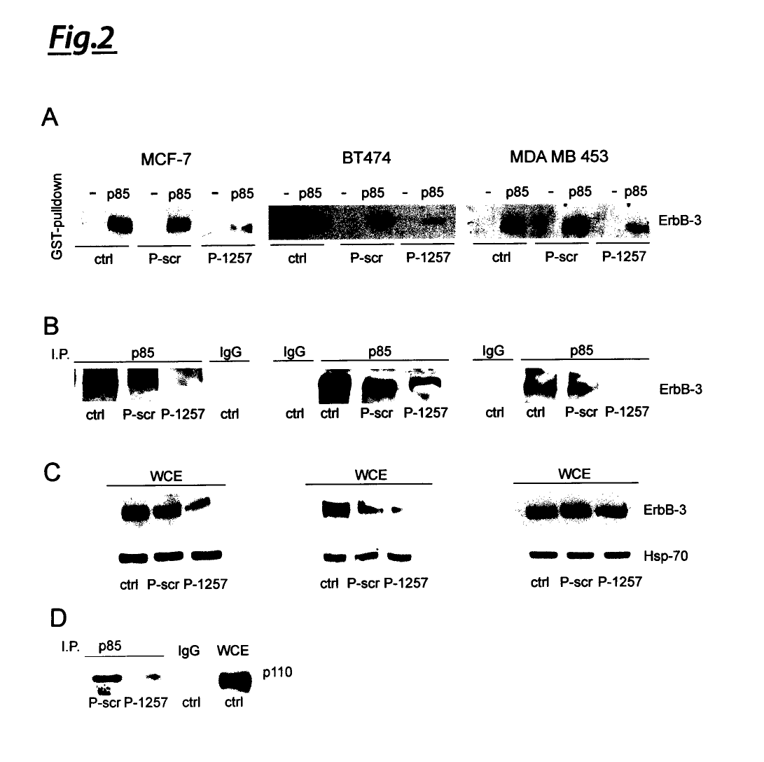

The authors of the present invention demonstrated that HER3 depletion, through RNA interference, by inhibiting PI3K activity, results in apoptosis of mammary tumour cells favouring the response to hormonal treatment with Tamoxifene (13). Moreover, it has been demonstrated that HER3 depletion in MCF7 and BT474 mammary tumour cells, through RNA interference, favours also the response to Herceptin treatment FIG. 1 (to be published data). As shown in FIG. 1, HER3 depletion inhibits MAPK and PI3K activities (the latter measured as phosphorylation levels of Akt, PI3K downstream kinase) and results in apoptosis, as demonstrated through the reduction of total PARP levels (FIGS. 1A, B, and C). The addition of Herceptin to culture medium, in order to mimic a combination therapy, induces an apoptosis increase and results in the complete PARP degradation indicating that HER3 depletion makes cells more vulnerable to Herceptin treatment (FIGS. 1B, and C). This result supports initial hypothesis suggesting that HER3, by activation PI3K mediated cell survival pathway, makes cells more resistant to Herceptin treatment.

This result induced the authors to study an alternative strategy to RNA interference mechanism since, up to now, the latter cannot be clinically used.

A previous study carried out by Dr. Koland team established that HER3 cytoplasmic domain comprises six binding sites (YXXM) for p85 SH2 domains (14). The same team had established that HER3 contains a proline rich region forming a consensus motif in order to bind p85 SH2 domains. But the study results had established that YXXM phosphorylated motifs of HER3 domain were mainly and principally responsible of HER3 and p85 interaction.

Successively Dr. Taiji team disclosed a sequence set of phospho-peptides corresponding to phosphorylated domains of HER3 cytoplasmic tail suitable to bind specifically p85 SH2 domains in N-terminal molecule portion (15). Both the studies have been carried out only aiming to study protein-protein interactions and never had a continuation aiming to use peptides, according to their design, for therapeutic or other purposes.

The Authors of the present invention have now found that a specific phosphopeptide, selected from above mentioned ones, is suitable to inhibit the binding of HER3 to p85, PI13K kinase regulating sub-unit, and therefore to inhibit the activity of said kinase. More particularly, the administration of phosphopeptide according to the invention is suitable to block HER3/p85 interaction, in HER2 hyper-expressing metastatic mammary tumour cells resulting in apoptosis and, further, enhances the response to Trastuzumab treatment (Herceptin®) both in vitro and in vivo.

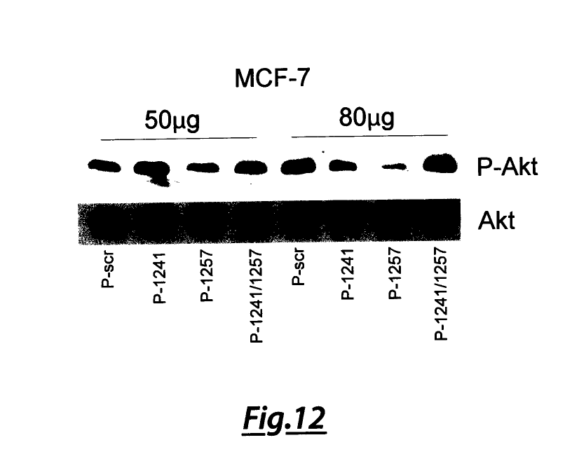

Out of eight peptides designed and tested by the above reported team, 1257:RDGGGPGGDpYAAMGACPA (SEQ ID NO:1) and 1241:PTAGTTPDEDpYEYMNRQR (SEQ ID NO: 5) are used for preliminary experiments. The choice of said two peptides resulted from the fact that both have very high association (Ka) and low dissociation constants (Kd), respectively. The two peptides have been used at various dosages, as shown in FIG. 12, in order to evaluate the ability thereof to inhibit PI3K activity measured as inhibition level of Akt phosphorylation. Obtained results demonstrate clearly that 1241 phosphopeptide is not suitable to remarkably inhibit PI3K activity when compared to high efficiency shown by 1257 peptide. The result could be attributable to the fact that 1241 peptide, although binds p85 with high efficiency (Ka 824), has a Kd (12.9) higher than 1257 peptide (5.17). Therefore it could be assumed that 1257 peptide remaining bound to p85 sub-unit longer, is more efficient from a biological point of view. Therefore, only pY1257 phosphopeptide: RDGGGPGGDpYAAMGACPA (SEQ ID NO: 1) (having high association and low dissociation constants referring to p85) displayed an optimal effectiveness from the biological point of view. For all the tests, both in in vitro and in vivo, scramble (scr), i.e. not specific, phosphopeptide has been used as control.

Therefore it is a specific object of the present invention a phosphopeptide consisting of the following sequence:

RDGGGPGGDpYAAMGACPA (SEQ ID NO: 1), or a nucleotide sequence encoding for said phosphopeptide or a vector comprising said nucleotide sequence, for use in medical field.

Moreover, the present invention concerns a phosphopeptide consisting of the following sequence:

RDGGGPGGDpYAAMGACPA (SEQ ID NO: 1), or a nucleotide sequence encoding for said phosphopeptide or a vector comprising said nucleotide sequence, for use in the treatment of tumours selected from solid, primary or metastatic, HER2 and HER3 hyper-expressing and having elevated PI3K activity tumours or primary or metastatic, positive for BRAF oncogene expression, melanomas.

Those skilled in the art know the methods in order hyper-expression of above said genes (13,18) to be detected in patient tumours thus allowing a specific therapy directed against specific tumour type to be carried out.

Among thus treatable tumours mammary ones can be mentioned, in addition to above specified melanomas displaying high levels of PI3K activity and in vitro responsive to the peptide. Phosphopeptide for use as above described can be conjugated to tumour targeting peptides, for example a N-terminal peptide suitable to bind MSH receptor, in order human melanomas to be targeted, and/or to be conjugated to anti-neoplastic drugs.

A further object of the present invention comprises pharmaceutical combinations of phosphopeptide consisting of the following sequence:

RDGGGPGGDpYAAMGACPA (SEQ ID NO: 1), or a nucleotide sequence encoding for said phosphopeptide or a vector comprising said nucleotide sequence, with one or more anti-tumour active principles. The latter can be selected from the group consisting of Trastuzumab, Anthracyclines with or without Docetaxel, Doxorubicin, Cyclophosphamide, Docetaxel, Carboplatin, Paclitaxel. Moreover, phosphopeptide could also be administered in Tamoxifene hormonal therapy and in combination with Bevacizumab humanized monoclonal antibody. As to very extended, operable or not, melanomas it could be administered by electroporation in combination with new generation drugs such as: PLX4720, RG7204 (also known as PLX4032), GSK2118436, and Farnesyl transferase inhibitor.

All these new generation compounds are used in order to inhibit the activity of NRAS or BRAF oncogenes characterizing most melanomas. BRAF oncogene expressing melanomas are most aggressive and have high levels of PI3K activity.

Some mammary tumours are treated with Tamoxifene, but approximately 12% of these tumours goes in progression due to PI3K high activity and levels of HER3 expression. Inventive phosphopeptide combination therapy would be therefore useful for these tumours.

The present invention further concerns pharmaceutical combinations as above defined for simultaneous, separated or delayed (time-spaced) use of said phosphopeptide, or nucleotide sequence or vector, and one or more anti-tumour active principles for therapy of above mentioned tumours.

In addition the invention concerns pharmaceutical combinations as above defined for use in the treatment of tumours selected from solid, primary or metastatic, HER2 and HER3 hyper-expressing and having elevated PI3K activity tumours or primary or metastatic, positive for BRAF oncogene expression, melanomas. As above said, the tumour can be mammary one, in addition to above mentioned melanomas.

The present invention concerns, moreover, pharmaceutical compositions comprising or consisting of phosphopeptide, or nucleotide sequence or vector, or as above defined combinations, like active principles, in combination with one or more pharmaceutically acceptable excipients and/or adjuvants.

Phosphopeptide according to the invention has been included among liposomes to be used for treatment of metastases. In order to favour the therapeutic target, the external membrane of 1257 phosphopeptide containing liposomes can be conjugated to an anti-tumour antibody. Examples of anti-tumour antibodies suitable to be conjugated with said phosphopeptide are the following: anti-HER2 Trastuzumab antibody (Herceptin) used for HER2 very positive mammary tumours; anti-HER1/HER2 Lapatinib used for HER2 positive and hormonal receptor positive mammary tumours, preferentially administered to menopause women together with aromatase inhibitor; anti-HER2/HER3 Pertuzumab used for HER2/HER3 expressing mammary tumours and often administered in combination with Trastuzumab for early stage tumours; anti-HER1 Cetuximab, Erlotinib and Gefitini, antibodies used for lung, pancreas, colon-rectum, breast, genitourinary tract, glioblastomas, cervix-facial district tumours; anti-c-Kit Imatinib used for inoperable gastro-enteric tumours, also named GIST, anti-VEFGR1 and VEFGR2 Bevacizumab (also named Avastin), directed against type 1 and 2 vascular endothelial growth factor receptor occurring on endothelial cells and therefore inhibiting tumour vascularization; anti-VEGF and PDGF Valatinib, that is soluble factors stimulating endothelial cell receptors, therefore, also in this case, inhibit vascularization.

The administration of peptide antibody is employed for tumours that, in addition to the expression of HER2, have HER3 high levels and therefore PI3k high activity. Almost all above listed tumours have PI3K high activity, although not surely activated only by HER3. Therefore, mammary tumours expressing high levels of HER2 and HER3 or BRAF melanomas expressing high levels of HER2 and HER3, as well as some lung and colon tumours, are among most eligible to be treated with combination therapy, that is peptide and antibody combination therapy. Above mentioned antibodies are generally administered in combination with chemotherapeutics.

In order to improve the therapeutic target, the phosphopeptide of the invention can be conjugated with TAT protein or RGD peptide and at the same time with anti-neoplastic drugs in order to be used also like a carrier.

A further object of the present invention comprises pharmaceutical compositions for use in treatment of tumours selected from solid, primary or metastatic, HER2 and HER3 hyper-expressing and having elevated

PI3K activity tumours or primary or metastatic, positive for BRAF oncogene expression, melanomas. As above said, in addition to above said melanomas the tumour can be mammary tumour.

The present invention now will be described, by an illustrative and not limitative way, according to preferred embodiments thereof, with particular reference to enclosed drawings wherein:

FIG. 1A shows: the inhibition of Akt phosphorylation in MCF7 and BT474 cell lines as determined through HER3 expression interference in the absence and presence of Herceptin compared to negative controls. FIG. 1B, C: apoptosis induction measured as increase of cell death and total PARP degradation compared to controls.

FIG. 2A,B shows: the efficiency of 1257 phosphopeptide, both in vitro (A: GST-pull) and in vivo (B: Immunoprecipitation), in inhibiting HER3 and p85 interaction compared to scr control and parental cells without peptide. In FIG. 2C total levels of HER3 and Hsp 70 as loading control are reported. FIG. 1D (Immunoprecipitation) shows the efficiency of 1257 phosphopeptide in inhibiting p85 and p110 (PI3K catalytic sub-unit) in vivo interaction.

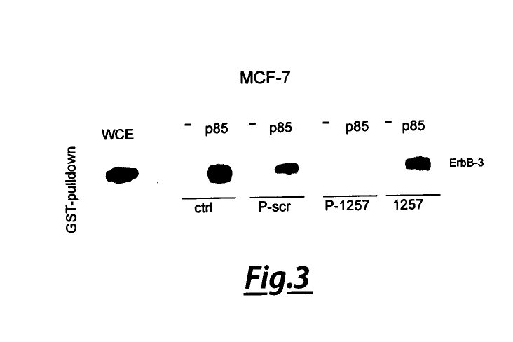

FIG. 3 shows: (GST-pull), according to data reported by Suenaga et al., not phosphorylated form of 1257 peptide does not interact with p85 SH2 domain because it does not inhibit p85 and ErbB-3 binding.

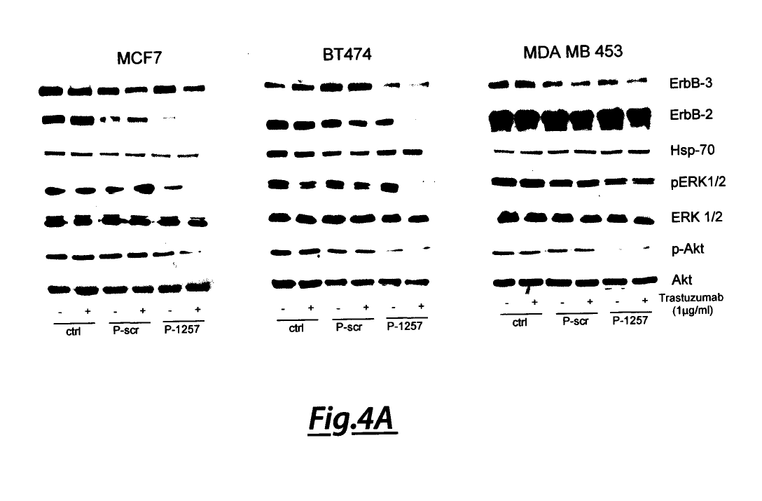

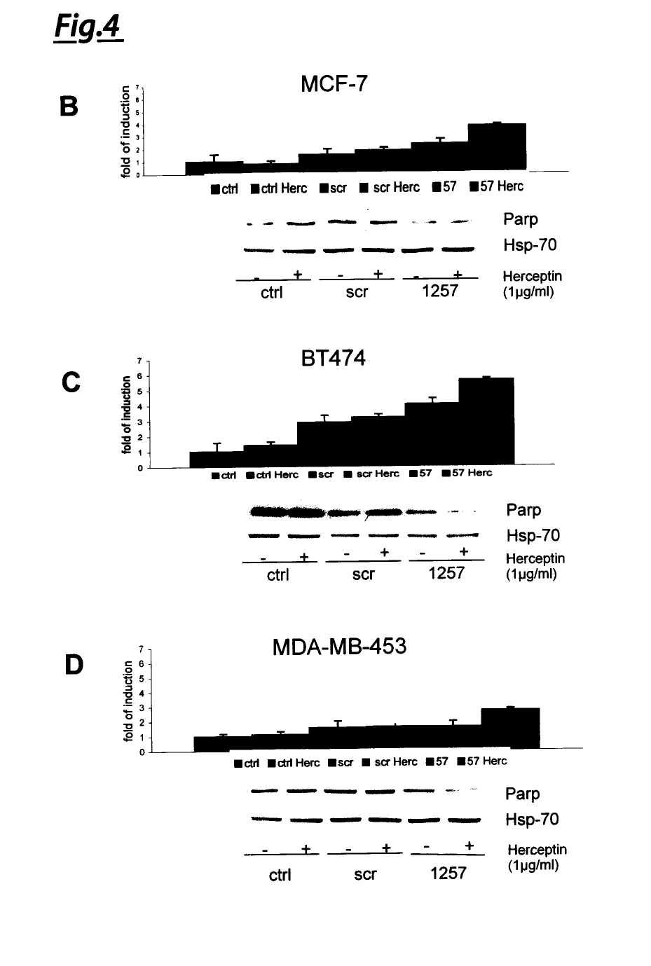

FIG. 4A shows: the inhibition of ERK1/2 and Akt phosphorylation levels and the reduction of HER3 and HER2 expression levels after transfection with 1257 peptide in the absence of and combination with Herceptin compared to controls in MCF7, BT474, and MDA-MD 453 cell lines. FIG. 4B, C, D: apoptosis induction reported as increase of cell death (referring to upper panel) and total PARP degradation (WB lower panels) for cells transfected with 1257 phosphopeptide in the absence and combination with Herceptin compared to negative controls.

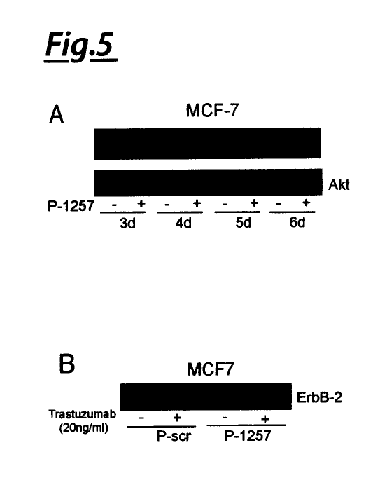

FIG. 5A shows: 1257 phosphopeptide inhibits P-Akt activity up to 6 days after the transfection indicating high stability of molecule. FIG. 5B: 1257 phosphopeptide causes high reduction of ErbB-3 levels (FIG. 2C) and this favours ErbB-2 homodimer increase as evaluated using non reducing experimental conditions. The addition of Herceptin results in full internalization of not longer membrane occurring receptor.

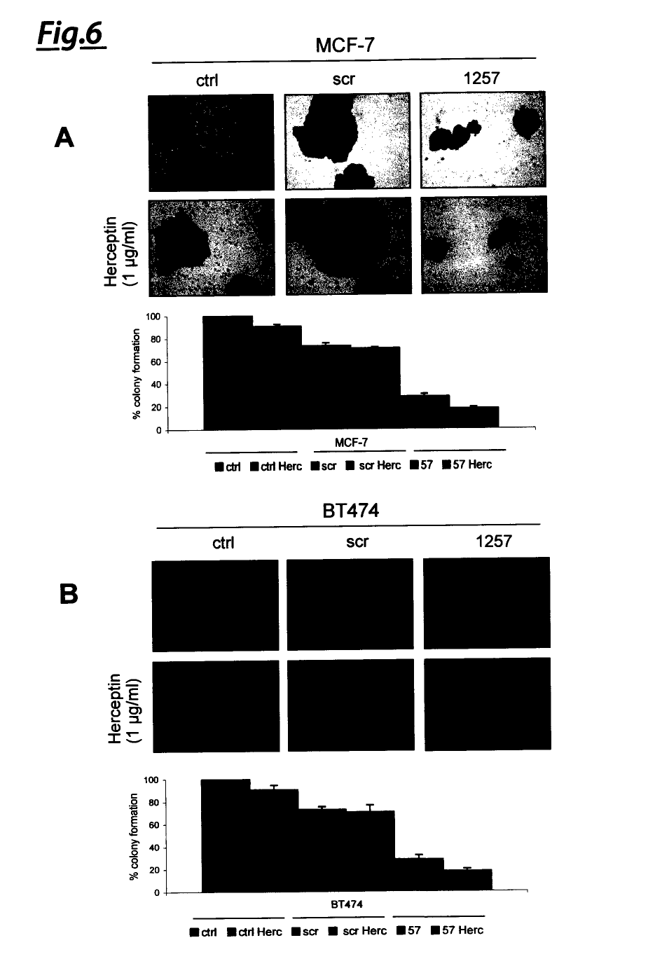

FIG. 6A, B shows: the inhibition of growth ability in anchorage-independence of MCF7 and BT474 cell lines after transfection with 1257 phosphopeptide in the absence and combination with Herceptin compared to negative controls (upper panels). The reduction of the colony number is plotted together with standard deviation (lower panels) compared to negative controls.

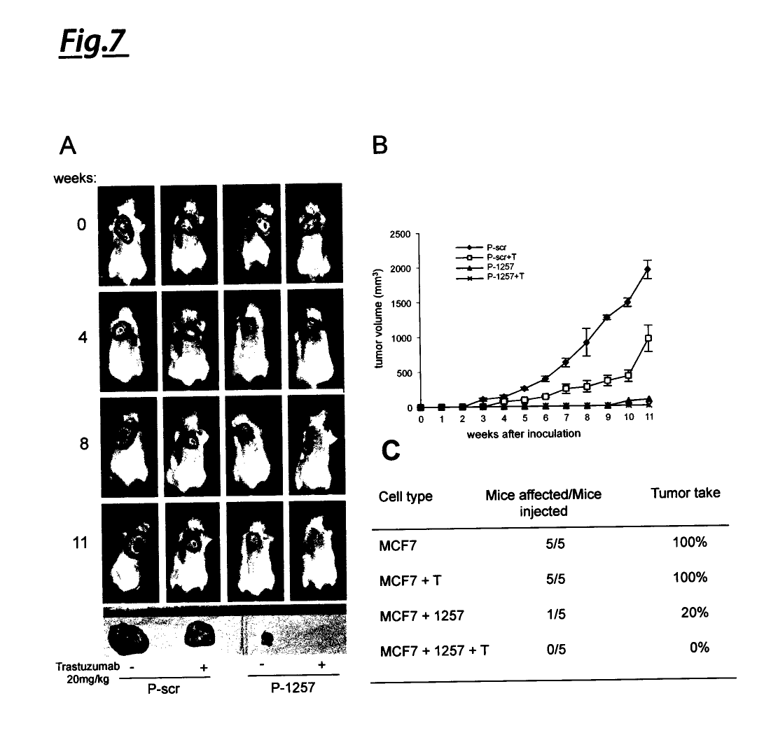

FIG. 7A, B, C shows: in vivo growth of pGL4.51-Luc stably transduced and 1257 or scr phosphopeptide transfected MCF7 cell line inoculated subcutaneously in Hercetin treated and not treated SCID mice. Tumour growth has been measured using bioluminescence, calibration and engraftment as reported in (A), (B), and (C), respectively.

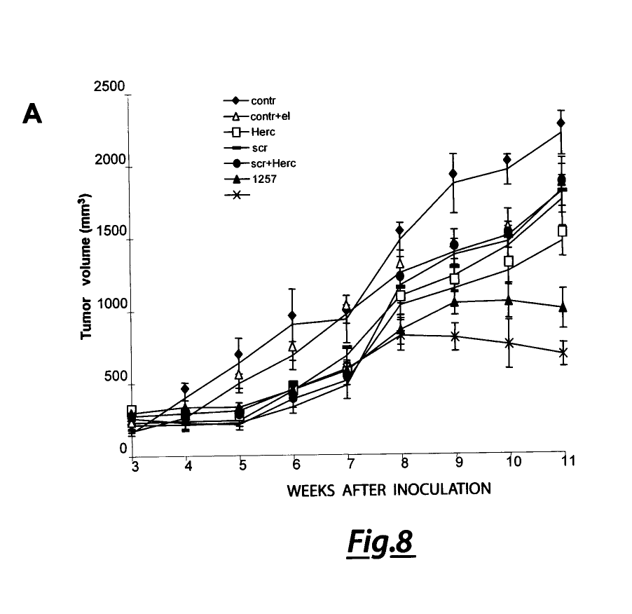

FIG. 8A shows: the in vivo growth, measured using calibration, of MCF7 cell line inoculated subcutaneously in SCID mice. Administration of 1257 or scr phosphopeptides by intra-tumour injection followed by electroporation in the presence or absence of Herceptin treatment. FIG. 8B: photographs of mice and respective tumours at the end of the treatments.

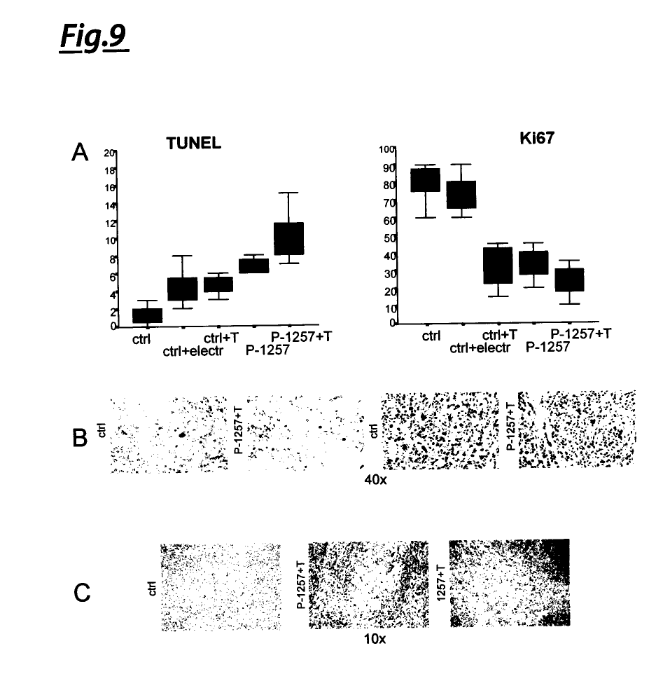

FIG. 9 shows: immunohistochemical tests in order to measure apoptosis levels (A) by TUNNEL assay and (B) proliferation levels by Ki67 assay in control and 1257 phosphopeptide electroporation treated tumours in the absence and presence of Herceptin greatment. C: eosin-hematoxylin staining to detect the necrosis occurrence within in vivo 1257 phosphopeptide electroporation and Herceptin treated tumours.

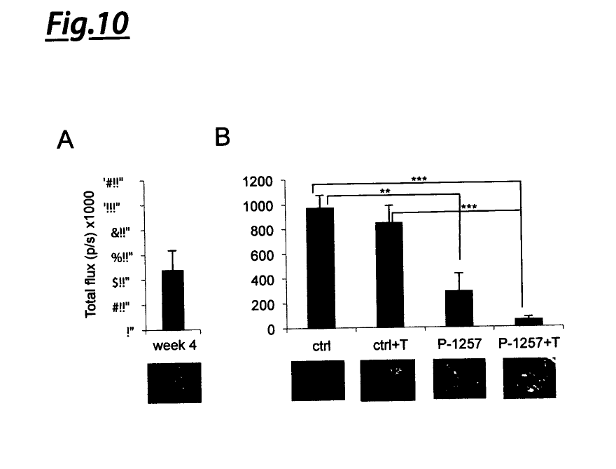

FIG. 10 shows: (A, B upper panel): (A) artificial metastasis, measured by bioluminescence, occurring in mouse lungs 4 weeks after tumour cell inoculation into the tail vein. (B): artificial metastasis, 7 weeks after above said inoculation and after treatment with empty or 1257 phosphopeptide containing liposomes in the absence and presence of Herceptin treatment. (A and B lower panel) shows lung bioluminescence at the end of the treatment.

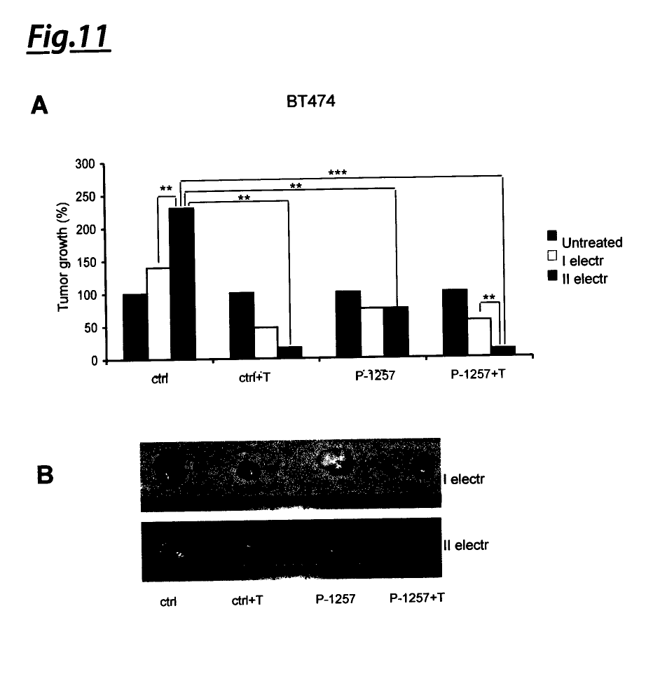

FIG. 11A shows: the in vivo tumour growth curve obtained by inoculation of Herceptin treatment responsive BT474 cells. The administration of 1257 or scr phosphopeptide by intra-tumour injection followed by electroporation in the presence or absence of Herceptin treatment. B: tumour photographs after the first and final treatment.

FIG. 12 shows Akt phosphorylation levels in MCF7 cells after two peptide transfection at different doses. 1257 phosphopeptide remarkably inhibits Akt phosphorylation. Combined 41/57 administration has been tried, but it proved to be not efficient.

### EXAMPLE 1

### In Vivo and Vitro Study about Biological and Biochemical Effects of Peptide According to the Invention in MCF7, BT474 and MDA-MB-453 Cell Lines

Materials and Methods

Cell lines, transfections, and reagents. MCF7 and MDA-MB-453 human mammary carcinoma cell lines have been supplied from the American Type Culture Collection (ATCC) (Manassas, Va.), and cultured in RPMI culture media supplemented with 10% FBS, 1% penicillin/streptomycin and 1% glutamine (Invitrogen, Milan, IT). BT474 cell line, supplied from ATCC, was cultured in as above supplemented DMEM media. The cells have been peptide transfected according to Lipofectamine (Invitrogen) standard method. Phosphatidylcholine (PC; 98% pure), cholesterol (the Chol; 99% pure) and 1,2-distearoyl-sn-glicero-3-phosphoethanolamine-N-[carbonyl-methoxy (polyethylene glycol) 2000] (ammonium salt) (mPeg2000-DSPE) have been obtained from Avant Polar Lipids (Albaster, Ala.). Liposomes have been extruded using Mixer-extruded obtained from Avant Polar Lipids.

Antibodies. Rabbit anti-phospho-ser Akt (#9271) and total anti-Akt (#9272), anti-phospho-ERK (#9101L) and total anti-ERK (#9102), and anti-PARP (#9542) and anti-p110a (#4255) antibodies have been supplied from Cell Signaling (Milan, IT). Rabbit anti-ErbB2 (#554299) and anti-ErbB-3 (sc-285) and mouse anti-Hsp-70 (N27F34) antibodies have been supplied from BD Biosciences and Stressgen (Milan, IT) and Santa Cruz Biotechnology (Milan, IT), respectively. Anti-rabbit and anti-mouse, HRP-conjugated secondary antibodies have been supplied from Bio-Rad (Milan, Italy). These antibodies have been used in Blot Western analysis.

Rabbit anti-p85 (#06-496) antibody used in immunoprecipitation assay has been supplied from Cell Signaling Company. Anti-Ki67 (#MIB-1) antibody has been supplied from Dako (Milan, Italy).

RNA interference. In order to interfere with ErbB3 expression, MCF7 and BT474 cell lines have been transiently transfected, using Transit-TKO (Mirus, Madison, Wis.) reagent kit, with double-strand RNA specific and control oligonucleotides, according to the supplier instructions.

Specific oligonucleotide has the following sequence:

(SEQ ID NO: 3)
5′-GCUCUACGAGAGGUGUGAG-3′
(SEQ ID NO: 6)
5′-CUCACACCUCUCGUAGAGC-3′

wherein at 3′ of each filament are inserted, during the synthesis, two thymine (T) bases for oligonucleotide stabilization.

Control oligonucleotide has the following sequence:

(SEQ ID NO: 4)
5′-GCGCGCAACUCUACCUCUA-3′
(SEQ ID NO: 7)
5′-UAGAGGUAGAGUUGCGCGC-3′

wherein at 3′ of each filament are inserted, during the synthesis, two thymine (T) bases for oligonucleotide stabilization.

Oligonucleotides have been synthesized by Oligoengine Inc. (Seattle, Wash.).

Peptide transfection. In order to inhibit the p85 and ErbB3 interaction, MCF7, BT474 and MDA-MB-453 cell lines have been transfected using 80, 100, and 180 μg, respectively, of the following phosphopeptides:

pY1257 phosphopeptide:
(SEQ ID NO: 1)
RDGGGPGGDpYAAMGACPA
or
scr phosphopeptide:
(SEQ ID NO: 2)
PYQMRpYNADTDGERTTEP

using Lipofectamine 2000 reagent (Invitrogen, Milan, IT), according to the supplier instructions. Not phosphorylated 1257 peptide has been used as further negative control. The peptides have been synthesized by INBIOS (Naples, IT).

Treatments and lmmunodecorations. ErbB3 interfered or peptide transfected MCF7, BT474 and MDA-MB-453 cell lines have been treated with Herceptin at 1 pg/ml for 40 hours. Briefly the cells have been plated at 5×10⁵ concentration in 60 mm plates. After 24 hours the cells have been transfected and three hours after the transfection treated with Herceptin or ethanol, used as control. At the end of the treatment the cells have been lysed with RIPA buffer [150 mM NaCl, 1% NP-40, 0,5% sodium deoxycholate (DOC), 0.1% SDS, 50 mM TrisHCl (pH 8), 1 mM PMSF, 1 mM EGTA, 50 mM NaF, 50 mM Na₃VO₄ and protease inhibitors (Roche, Milano, IT)] in order to analyze the expression levels of phosphorylated and total ErbB-3, ErbB-2, ERK and HSP-70 proteins. In order to analyze the levels of Akt phosphorylation and expression, the cells have been lysed in NP-40 buffer [1% NP-40, 10% glycerol, 137 mM NaCl, 20 mM TrisHCl (pH 7.4), 50 mM NaF, 1 mM PMSF, 5 mM Na₃VO₄, protease inhibitors (Roche, Milano, IT)]. Cell lysates have been incubated on ice for 20 minutes and centrifuged at 14000 rpm for 20 minutes. RIPA buffer obtained cell extracts have been boiled for 5 minutes at 95° C., while NP40 buffer lysed samples have been boiled at 65° C. for 5 minutes. Samples have been subjected to SDS-PAGE and transferred on nitrocellulose filters (Bio-Rad, Milan, IT). Then the filters have been incubated with interest antibodies, washed and incubated with specific secondary horse radish peroxidase (HRP) conjugated antibodies. After thorough washing, nitrocellulose peroxidase activity has been detected by chemiluminescence (Lite-blot-Euroclone, Milano, IT)

Cell death and Apoptosis. MCF7, BT474 and MDA-MB-453 cell lines have been plated at 5×10⁵ concentration in 60 mm plates. On subsequent day the cells have been transfected with ErbB3 specific interference oligonucleotides or peptides. 3 hours after the transfection the cells have been treated with Herceptin (1 μg/ml) for 40 hours. The cells have been washed two times with cold PBS and then trypsin collected. The evaluation of cellular viability has been carried out by means of Trypan Blue exclusion. The use of anti-PARP antibody in Western Blotting analysis allowed apoptosis to be evaluated. Briefly the cells have been lysed in RIPA buffer [150 mM NaCl, 1% NP-40, 0,5% sodium deoxycholate (DOC), 0.1% SDS, 50 mM TrisHCl (pH 8), 1 mM PMSF, 1 mM EGTA, 50 mM NaF, 50 mM Na₃VO₄ and protease inhibitors (Roche, Milano, IT)]. The samples have been boiled for 5 minutes at 95° C., resolved using SDS-PAGE, transferred on nitrocellulose and blotted using anti-PARP antibody. The tumour cell death analyzed by immunohistochemistry have been obtained using TUNEL assay.

GST pull-down. MCF-7, BT474 and MDA-MB-453 cell lines have been washed 2 times with cold PBS and extracted with lysis buffer [(50 mM Tris HCl (pH 7.4), 250 mM NaCl, 0,1% Triton X-100, 5 mM EDTA, 50 mM NaF, 1 mM PMSF, 5 mM Na₃VO₄ and 50 mmol/L of protease inhibitors (Roche, Milano, IT)]. After 20 minute ice-cold incubation, the lysates have been clarified by centrifugation at 14000 rpm for 20 minutes. The cellular extracts (1 mg sample) have been incubated for 4 hours at 4° C. in the presence of GST bound gluthatione-agarose beads or GST-p85 fusion protein (kindly supplied by doctor S. Giordano), in the presence or absence of 1257 phosphopeptide or scr peptide used as control. Then the interaction complexes have been washed six times with lysis buffer and subsequently beads eluted using Laemli Buffer 2× [(140 mM SDS, 432 mM Glycerol, 1.6 mM Bromophenol blue, 120 mM Tris HCl (pH 6.8), 8% β-mercaptoethanol)]. The proteins have been separated using 8% SDS-PAGE and transferred on nitrocellulose filter. The filter has been then immunodecorated with anti ErbB3 antibody.

Immunoprecipitation. MCF7, BT474 and MDA-MB-453 cell lines have been plated at 5×10⁵ concentration in 60 mm plates. On subsequent day cells have been transfected with 1257 phosphopeptide and contrrol phosphopeptide. After 40 hours the cells have been washed two times with cold PBS and extracted with lysis buffer [(50 mM Tris HCl (pH 7.4), 250 mM NaCl, 0.1% Triton X-100, 5 mM EDTA, 50 mM NaF, 1 mM PMSF, 5 mM Na₃VO₄ e 50 mmol/L protease inhibitors (Roche, Milan, IT)]. Whole cell lysates have been imunoprecipitated with anti-p85 antibody (#06-496) in the presence of protein G (Pierce, Milan, IT). Whole proteins and immunocomplexes then have been subjected to 8% SDS-PAGE and transferred on nitrocellulose filter. The proteins have been detected by immunodecoration using anti-ErbB3 and anti-p110α antibodies.

Soft agar. Control and 1257 phosphopeptide transfected MCF7 and BT474 cells, in the presence and absence of Herceptin, have been plated on soft-agar in order to evaluate anchorage-independent growth under different conditions, as previously described (19). Briefly cells (1×10⁴) have been re-suspended in 5 ml of 0.3% agar in RPMI or DMEM supplemented with 10% FCS, and quickly stained on a solid layer of 1.2% agar in DMEM/RPMI. Cultures have been carried out for 4 weeks. Then colonies have been stained with viable colony specific neutral red solution (Sigma, Milan, IT), and then counted. Photographies have been taken using Leica DM IRE2 microscope and Leica FW4000 software (Leica Microsystems, Milan, IT).

Preparation of liposomes. Unilamellar vesicles consisting of PC/Chol/mPEG-2000-DSPE (47/47/6% molar ratio) have been prepared by means of lipid film hydration. Briefly, the composition of phospholipids (PC), (Chol and MPEG-2000-DSPE), has been dissolved in a small volume of methanol/chloroform (50/50) mixture. Successively the solvent allowed to be evaporated under mild nitrogen flow. For the preparation of empty liposomes, the lipid film has been hydrated with 5 ml of HBS isotonic buffer (140 mM NaCl and 10 mM Hepes) using vortex mixer for 1 hr. For loading liposomes with 1257 phosphopeptide the freezing-thawing cyclic method has been used. The lipid film has been hydrated with 2.5 m of HBS buffer. The empty liposome suspension has been mixed with 2.5 ml of a solution containing 500 M of 1257 phosphopeptide in HBS buffer. Then the solution has been frozen at −196° C. in liquid nitrogen and thawed over 5 minutes at 30° C. in thermostated bath for 10 consecutive cycles in order to allow the peptide penetration into liposomes. After each cycle the suspension has been stirred for 60 sec using a vortex mixer. At the end of the cycles, the liposome suspension has been ultracentrifuged two times at 34000 rpm for 4 hr using Beckman SW55 centrifuge in order to remove free 1257 phosphopetide.

Determination of encapsulation efficiency. The encapsulation efficiency has been estimated using RP-HPLC method (HP Agilent with apparatus series No. 1100) (Santa Clara, Calif., USA). Samples have been loaded on 4.6 mm×250 mm Phenomenex C18 (Torrance, Calif.) column with average flow rate of 1.0 ml/min⁻¹. 0.1% TFA in H₂O (a) and 0.1% TFA in CH₃CN (b) has been used as solvent. Columns have been eluted with two linear gradients at a flow rate of 1.0 ml/min: i) 5 to 70% A in 30 min and then 70 to 95% B in 10 min. Liposomes have been quantified with DAD detector at 210 nm visible wavelength. Absorbance calibration curve of peptide has been carried out in isotonic HBS buffer.

Xeno-grafts. In vivo tests have been carried out using 40 day old SCID female mice (Harlan Laboratories, Milan, IT). At day 0, animals have been totally anesthetized with intramuscular injection of 400 μg/animal (average weight 25 g) of Zoletil (Virbac) containing 0.12% Xylor (Xylazine), and transplanted subcutaneously (s.c.), in intrascapular mouse region with pellet of 17-h-extradiol (1.7 mg/sustained release pellet over 60 days, Innovative Research of America, Sarasota, Fla.). On the next day, 5×10⁶ proliferating and scr and 1257 peptide previously transfected, as above described, MCF7 or BT474 cells, have been re-suspended in 0.1 ml of Matrigel (BD Biosciences) and subcutaneously inoculated in each mouse. Herceptin treatment involved intraperitoneal injection of 250 ul of sterile PBS containing 500 ug of antibody, two times weekly. Tumour growth has been monitored two times weekly using calibration along two orthogonal axes: length (L) and depth (VV). The tumour volume (V) has been calculated using the formula V=L*(W2)/2. All the procedures relating to animals and treatment thereof have been carried out according institutional guidelines.

Generation of mammary tumour luminescent cells. Mammary tumour MCF7 cell lines stably transfected using pGL4.51 (luc2/CMV/Neo) expression vector, produced by Promega (Me, IT), have been transfected using scr or 1257 phosphopeptide (80 g×5×10⁴ cells/mouse) and after 30 h subcutaneously inoculated in SCID mice (5×10⁶ cells/mouse) in 200 μl (1:1 Matrigel: cell suspension in serum free medium). Mice have been in vivo imaged 20′ after inoculation and once weekly over 11 weeks. The animals have been anesthetized, as above reported, and subjected to intraperitoneal injection with 200 μl of luciferin (Caliper, Mi, IT); 10′ after incubation photon emission has been analyzed at 3′ minute exposure using Xenogen Ivis Lumina 2 machine. Growth of these cells has been monitored as reported in xenografts section.

Electroporation tumour therapy. MCF7 or BT474 cell xenografted SCID mice have been treated with 1257 peptide through electroporation using Square Electro Porator (CUY21; Nepagene) when the tumour had an average volume of 200 mm³. A dilute solution of 1257 peptide (75 ug) in 15 ul of sterile PBS has been directly injected in each xenograft, which subsequently has been electrode pulsed. PBS diluted scr peptide has been used as control, according to same procedure. As a further control we have analyzed two groups of not peptide injected animals, except that one of two was subjected to electroporation. Therapy has been repeated once weekly, and tumour size monitored up to 70 days. Herceptin® treatment has been carried out two times weekly with antibody intraperitoneal injection, as above described.

Tumour size has been measured at regular intervals (alternate days) using a caliber and tumour volume has been calculated as above described. In order to minimize animal pain during the electroporation, tests have been carried out under total anesthesia. All the procedures concerning animals and treatment thereof have been carried out according to institutional guidelines.

All the tests have been repeated on a group of at least 8 animals under each experimental condition set and repeated at least twice. In vivo data statistical significance has been evaluated using Student Test (P<0.05).

### Experimental Part

The sequence of two peptides synthesized and used in below reported studies is:

pY1257 phosphopeptide:
(SEQ ID NO: 1)
RDGGGPGGDpYAAMGACPA,
pYscr phosphopeptide:
(SEQ ID NO: 2)
PYQMRpYNADTDGERTTEP.

Both >90% pure phosphopeptides have been synthesised by INBIOS (IT) company.

Tests carried out in order to verify the effectiveness of this peptide both from biochemical and biological point of view have been the following ones:

As in FIG. 2A is apparent that 1257 phosphopeptide effectively in vitro binds p85 sub-unit thus inhibiting HER3 receptor binding. Using pull-down tests it has been proved that said peptide is suitable in vitro to bind GST-N-SH2p85 fusion protein, that is GST protein (gluthatione-transferase) in “frame” containing SH2 domains in N-terminal portion of p85 sub-unit. In this regard, cell lysates from three different mammary tumour cell lines (BT474, MCF7 and MDA-MB-453 considered poor or not responsive Herceptin teatment) have been used. This experiment proved that 1257 phosphopeptide efficiently binds fusion protein because the latter one, in presence of peptide, is not suitable to recruit HER3 receptor. As expected, for control lysates in the presence of pYscr peptide the binding is as efficient as in the absence of peptide indicating that 1257 phosphopeptide inhibits in specific way HER3 and p85 SH2 domain binding.

FIG. 2B shows the peptide effectiveness in inhibiting p85 and HER3 interaction in vivo. The peptide has been transfected using Lipofectamine in all three cell lines. 48 hr after transfection cells have been lysed and using immunoprecipitation tests with sub-unit p85 directed antibody and western blot assays with HER3 receptor directed antibody it has been verified that phosphopeptide inhibits effectively p85 and HER3 interaction also in vivo. In FIG. 2D an analogous experiment carried out to verify that 1257 phosphopeptide inhibits p85 from interacting with p110a, PI3K catalytic sub-unit, is shown. This binding is required in order PI3K kinase to be activated.

FIG. 3 shows the result of an experiment carried out in order to confirm 1257 phosphopeptide specificity in inhibiting PI3K activity. To be sure about 1257 phosphopeptide specificity said not phosphorylated 1257 peptide has been transfected using Lipofectamina in MCF7 cells. 48 hr after transfection cells have been lysed and using western blot assays we have verified that not phosphorylated 1257 peptide is unable to inhibit Akt phosphorylation.

Then the activation condition of intracytoplasmic signal pathways downstream of HER2 and HER3 receptors has been verified. As reported in FIG. 4A, results show that the transfection with the peptide results in inhibition the phosphorylation of Akt, kinase directly downstream of PI3K, and also inhibition of MAPK phosphorylation for all used cell lines. Both the pathways are further inhibited or deleted if the cells are also treated with Herceptin. The treatment with scr peptide, alone or in combination with Herceptin, does not result in any effect on both activation pathways. Further we have noticed that treatment with 1257 phosphopeptide determines a decrease of: i) total level of HER3, resulting from reduced synthesis due to inhibition of PI3K activity as previously demonstrated (16) (FIGS. 1C and 4A); ii) total level of HER2, according to a large number of literature data wherein it has been demonstrated that Herceptin results in the internalization and degradation of HER2. The decrease of expression levels for both receptors is poorly appreciable in MDA-MB-453 cell lines because in these cells phosphorylation level for both receptors is low.

After the verification of peptide effectiveness from the biochemical point of view, it has been attempted to understand the in vitro biological effect of the treatment with peptide alone and/or in combination with Herceptin®. Obtained results show clearly that phosphopeptide, in comparison to three parental or scr peptide transfected cell lines, induces cell death by apoptosis measured as: a) induction times of cell death; b) expression levels of processed PARP. Apoptosis induction levels, measured in comparison to control samples, show that: in MCF7 cells, not responsive to Herceptin® treatment, phosphopeptide induces death 3 times higher than controls. Herceptin® combination treatment results in cell death increase up to 4 times; in partially responsive Herceptin® treatment BT474 cells, phosphopeptide induces a cell death 4 times higher than controls, and in the presence of Herceptin® it increases up 6 times, in not responsive Herceptin® treatment MDA-MB-453 cells the combination treatment induces a cell death 2 times higher than the controls (FIG. 4B, C, D, respectively).

Since the inhibition of PI3K and MAPK signal pathways caused by 1257 phosphopeptide were very strong, we have estimated the phosphopeptide stability inside of cell. As shown in FIG. 5A, 1257 phosphopeptide is suitable to inhibit Akt phosphorylation up to 6 days after transfection thus supporting the hypothesis about an high stable molecule. Since 1257 phosphopeptide reduces ErbB-3 levels in membrane (see FIG. 2C) and favours the response to Herceptin treatment, we have verified ErbB2 homodimer levels. FIG. 5B shows that the transfection with 1257 phosphopeptide favours the formation of ErbB-2 homodimer, as analyzed under not reducing conditions. This result explains because in the presence of 1257 phosphopetide the ability of these cells to be responsive to Herceptin treatment is restored.

Then it has been attempted to understand whether the peptide is suitable to reduce the cell ability to grow under anchorage-independence conditions. To this purpose, MCF7 and BT474 control cells, or 1257 or scr phosphopeptide transfected cells have been plated on agar and allowed to grow for three weeks in the presence or absence of Herceptin®. As shown in FIG. 6, obtained results demonstrate that phosphopeptide reduces the growth (defined as colony largeness and number) ability under anchorage-independence conditions of both cell lines. Particularly, the phosphopeptide, compared to controls (parental or scr peptide transfected cells) results, for both cell lines, in a reduction of colony number up to 80% and up nearly 90% when Herceptin® is added to the culture. These colonies are very small and in some cases seem abortive suggesting that the data, in this last case, could be over-estimated.

Then it has been verified whether 1257 phosphopeptide was effective also in vivo. To this purpose immunocompromised SCID mice have been used. In the presence of oestrogen tablets, 5×10⁶ control cells transfected with scr peptide or transfected with 1257 peptide have been injected subcutaneously between mouse scapulae. In order to verify the viability of phosphopeptide transfected cells in animals we have generated a stable luciferase gene expressing MCF7 cell line (MCF7/Luc). Then MCF7/luc and 1257 and scr transfected cells have been inoculated s.c in the animals according to the following protocol:
8 animals inoculated with MCF7/peptide scr
8 animals inoculated with MCF7/peptide scr+Herceptin
8 animals inoculated with MCF7/1257 peptide
8 animals inoculated with MCF7/1257 peptide+Herceptin

FIGS. 7A, B, and C shows obtained results:
The tumours derived from MCF7 control cells transfected with scr peptide were calibratable three weeks after the inoculation and exponentially grown up to 11^{th} week at which time the animals have been sacrificed ulcerations being present (100% tumour take).
The tumours derived from the same cells, but treated with Herceptin, have shown a growth delay up to sixth week, then the growth was exponential up to about 12^{th} week and then control tumour similar (100% tumour take).
The tumours derived from the animals inoculated with MCF7 cells transfected with 1257 phosphopeptide were calibratable at about 10^{th} week, and the growth of still very small tumours did not display variations up to 16^{th} week at which time animals have been sacrificed. One out of five animals displayed tumour indicating a reduction of 80% of tumour take.
The animals inoculated with MCF7 cells transfected with 1257 phosphopeptide and treated with Herceptin have never shown tumour up 16^{th} week at which time the animals have been sacrificed (0% tumour take).

During the in vivo growth and after intraperitoneal inoculation of luciferin substrate these cells become luminescent and can be visualized using In Vivo Imaging machine (Xenogen Ivis Lumina 2) (17). As shown in FIG. 7A, data show that, after substrate inoculation, transplanted cells (controls or transfected with 1257 phosphopeptide) are all luminescent. Over the time luminescence increases for the controls and progressively decreases for cells transfected with 1257 phosphopeptide and combination therapy. Control and treated animals have been analyzed at regular intervals weekly up to 11^{th} week and 16^{th} week, respectively (FIG. 7A, B, C).

In an attempt to use an experimental approach mimicking human therapy, scr or 1257 phosphopeptides has been administered directly into tumour. The administration of the peptide has been carried out by injection and successive electroporation in order to allow the phosphopeptide penetration. This technique facilitating the drug absorption from the tumour is used for human mainly to treat superficial lesions like melanomas, skin tumours or cutaneous metastases, but currently it is also used for carcinomas during pre-operation sessions. The peptide administration has been carried out when the tumour volume was 200 mm³. The experiments have been carried out according to the following protocol:
8 animals inoculated with MCF7
8 animals inoculated with MCF7+Electroporation
8 animals inoculated with MCF7+Herceptin+Electroporation
8 animals inoculated with MCF7+scr phosphopeptide+Electroporation
8 animals inoculated with MCF7+scr phosphopeptide+Herceptin+Electroporation
8 animals inoculated with MCF7+1257 phosphopeptide+Electroporation
8 animals inoculated with MCF7+1257 phosphopeptide+Herceptin+Electroporation

The electroporation induces an immune response and for this reason it has been carried out also in control animals in order to reduce auto-generated background.

The results in FIGS. 8A and B show that the administration of 1257 phosphopeptide inhibits the growth of tumour (50%) compared to scr peptide inoculated and control not treated tumours (58%). The combination therapy in the presence of Herceptin® further inhibits growth of 1257 phosphopeptide (72%) inoculated tumours compared to control, scr peptide inoculated and Herceptin® treated tumours(66%).

In FIGS. 9A and B immunoistochemical analysis, using TUNNEL or anti-Ki67 assays, of tumours has pointed out that electroporation phosphopeptide treated tumours go in apoptosis and show a low proliferating index and both the biological effects are amplified when the animals are treated also with Herceptin. In FIG. 9C eosin-ematoxylin staining has evidenced some extended necrotic areas in tumours treated with combination therapy (1257 phosphopeptide and Herceptin).

These last data suggest that 1257 phosphopeptide has enormous potentiality for a future therapeutic application.

Since the object of the work is to inhibit or reduce the metastasis formation, it has been verified whether 1257 phosphopeptide was suitable not only to reduce the tumour, but also cell metastatic ability. To this purpose artificial metastases have been generated by vein inoculation of MCF7/luc cells in order to visualize tumours through luminescence. Four weeks after the inoculation when the pulmonary metastases were visible (FIG. 10A) the animals are treated for three (once weekly) consecutive weeks with empty or 1257 phosphopeptide containing liposomes. Half of each animal group has been treated also with Herceptin.

FIG. 10B, at the end of the experiment (7 weeks after vein inoculation of the tumour cells), shows that the animals treated with empty liposomes in the absence or presence of Herceptin have a strong luminescence indicating the presence of large metastases in some cases covering the whole pulmonary lobe. This result indicates that in the absence or presence of treatment with Herceptin a progression of lung metastasis occurs. On the contrary, the animals treated with liposome encapsulated 1257 phosphopeptide showed a strong reduction of metastases, and for combination treatment (liposomes/1257 and Herceptin) a nearly complete remission has been obtained. This result indicates that 1257 phosphopeptide as such is suitable to reduce already present metastases and that it is suitable to boost the Herceptin activity.

It has been then verified whether the treatment with 1257 phosphopeptide could have some effect on Herceptin responsive tumours. At this purpose BT474 cells have been inoculated subcutaneously in SCID mice and treated with scr or 1257 phosphopeptide by electroporation as above described.

The FIG. 11A: shows that the therapy with Herceptin, as expected, reduces very remarkably the tumour mass resulting from BT474 cells compared to not treated control (p≦0.001 Herceptin vs not treated). 1257 phosphopeptide treatment through electroporation strongly reduces the tumour mass compared to not treated control (p≦0.004 1257 phosphopeptide vs not treated) and the combination treatment efficiently reduces the tumour mass as Herceptin alone (p≦0.0001 1257 phosphopeptide+Herceptin vs not treated). Although in vivo data indicate that for Herceptin responsive cells 1257 phosphopeptide is not efficient as expected, data obtained from combination therapy indicate that this therapy reduces the tumour mass more effectively than Herceptin alone (p≦0.0001 vs p≦0.001) suggesting that 1257 phosphopeptide could improve the Herceptin response also in responsive tumours. FIG. 11B shows the largeness of the tumours at the end of the therapy.

These data are particularly important because demonstrate that the phosphopeptide, by inhibiting the PI3K survival signal pathway, makes the tumour more vulnerable and favours the response to therapy with Herceptin®.

### BIBLIOGRAPHY

1. Klapper L N, Kirschbaum M H, Sela M, Yarden Y. Biochemical and clinical implications of the ErbB/HER signaling network of growth factor receptors. Advances in cancer Res. 77:25-79, 2000.
2. Hynes N E, Lane H A. ERBB receptors and cancer: the complexity of target inhibitor. Nat Rev Cancer 5:341-54, 2005
3. Yarden Y. Biology of HER2 and its importance in breast cancer. Oncology 61:1-13, 2001.
4. Cooke T, Reeves J, Lanigan A, Stranton P. HER2 as prognostic and predective marker for breast cancer. Ann. Oncol. 12:S23-S28, 2001.
5. Slamon D J, Leyland-Jones B, Shak S et al., Use of chemotherapy plus a monoclonal antibody against HER2 for metastatic breast cancer that overexpresses HER2. N Engl J Med 344:783-92, 2001
6. Fresno Vara J A, Casado E, de Castro J, Cejas P, Belda-Iniesta C, Gonzales-Baron M. PI3K/Akt signaling pathway and cancer. Cancer Treat. Reviews 30:193-204, 2004.
7. Akt kinases in breast cancer and the results of adjuvant therapy. Breast Cancer Res. 5:R37-R44, 2003.
8. Berns K, Horlings H M, Hennessy B T, et al., A functional genetic approach identifies the PI3K pathway as a major determinant of trastuzumab resistance in breast cancer. Cancer Cell 12:395-402, 2007.
9. Wallasch C, Weiss F U, Niederfell G, et al. Heregulin-dependent regulation of HER2/neu oncogene by heterodimerization with HER3. Embo J. 14:4267-75, 1995.
10. Holbro T, Beerli R R, Maurer F et al. The ErbB2/ErbB3 heterodimer functions as an oncogenic unit: ErbB2 requires ErbB3 to drive breast tumor proliferation. Proc Natl Acad Sci USA 108:8933-8, 2003.
12. Cleary J M, Shapiro G I. Development of phosphoinositide-3 kinase pathway inhibitors for advanced cancer. Curr. Oncol. Rep. 12:87-94, 2010.
13. Folgiero V, Avetrani P, Bon G et al. Induction of ErbB-3 expression by alpha6beta4 integrin contributes to tamoxifen resistance in ERbeta1-negative breast cancinomas. PLoS ONE 13:e1592, 2008.
14. Hellyer N J, Cheng K, Koland J G. ErbB3 (HER3) interaction with p85 regulatory subunit
15. Suenaga A, Takada N, Hatakeyama M, et al. Novel mechanism of interaction of p85 subunit of phosphatidylinositol 3-kinase and ErbB3 receptor-derived phosphotyrosyl peptides. J. Biol. Chem. 280:1321-6, 2005.
16. Folgiero V, Bachelder R, Bon G, et al. The alpha6beta4 integrin can regulate ErbB-3 expression: implications for alpha6beta4 signaling and function. Cancer Res. 67:1645-52, 2007.
17. Signore A, Mather S J, Piaggio G, et al. Molecular imaging of inflammation/infection: nuclear medicine and optical imaging agents and methods. Chem Rev. 2010 May 12;110(5):3112-45.
18.Viros A., Fridlyand J., bauer J. et al. Improving Melanoma classification by integrating genetic and morphologic features. PLoS Medicine 5:e120, 2008.
19. Bon G, Folgiero V, Bossi G, et al. Loss of beta4 integrin subunit reduces the tumorigenicity of MCF7 mammay cells and causes apoptosis upon hormone deprivation. Clin. Cancer Res. 12:3280-7, 2006.

1. A medicament comprising a phosphopeptide consisting of sequence:
RDGGGPGGDpYAAMGACPA (SEQ ID NO: 1), or a nucleotide sequence encoding for said phosphopeptide or a vector comprising said nucleotide sequence.

2. A medicament comprising a phosphopeptide consisting of the following sequence:
RDGGGPGGDpYAAMGACPA (SEQ ID NO: 1), or a nucleotide sequence encoding for said phosphopeptide or a vector comprising said nucleotide sequence, in an amount suitable for treatment of a tumour selected from the group consisting of a solid tumour, a primary or metastatic tumour, a HER2 and HER3 hyper-expressing and having elevated PI3K activity tumour or a primary or metastatic tumor, a tumour positive for BRAF oncogene expression, and a melanoma.

3. The medicament according to claim 2, wherein the tumour is a mammary tumour.

4. The medicament according to claim 1, wherein said phosphopeptide is conjugated with a tumour targeting peptide and/or with an antineoplastic.

5. A pharmaceutical combination of a phosphopeptide consisting of sequence:
RDGGGPGGDpYAAMGACPA (SEQ ID NO: 1), or a nucleotide sequence encoding for said phosphopeptide or a vector comprising said nucleotide sequence, with one or more anti-tumour active principles.

6. The pharmaceutical combination according to claim 5, wherein the anti-tumour active principles are selected from the group consisting of trastuzumab, Anthracyclines with or without docetaxel, Doxorubicin, Cyclophosphamide, Docetaxel, Carboplatin, Paclitaxel, Tamoxifene, humanized monoclonal antibody Bevacizumab, PLX4720, GSK2118436 or Farnesyl transferase inhibitor.

7. The pharmaceutical combination of claim 5 formulated for simultaneous, separated or delayed use of said phosphopeptide, or nucleotide sequence or vector, and one or more anti-tumour active principles.

8. The pharmaceutical combination of claims 5-7 formulated for treatment of a tumour selected from the group consisting of a solid tumour, a primary or metastatic tumour, a HER2 and HER3 hyper-expressing and having elevated PI3K activity tumour or primary or metastatic tumour, a tumour positive for BRAF oncogene expression, and a melanoma.

9. The pharmaceutical combination according to claim 8, wherein the tumour is a mammary tumour.

10. A pharmaceutical composition comprising a phosphopeptide consisting of sequence RDGGGPGGDpYAAMGACPA (SEQ ID NO: 1), or a nucleotide sequence encoding for said phosphopeptide or a vector comprising said nucleotide sequence alone or in combination with one or more anti-tumour active principles, as active principles, in combination with one or more pharmaceutically acceptable excipients and/or adjuvants.

11. The pharmaceutical composition according to claim 10, wherein the composition is in form of liposomes.

12. The pharmaceutical composition according to claim 11, wherein the liposomes' membrane is conjugated with anti-tumour antibodies.

13. The pharmaceutical composition according to claim 10 formulated for treatment of tumours selected from the group consisting of a solid tumour, a primary or metastatic tumour, a HER2 and HER3 hyper-expressing and having elevated PI3K activity tumour or a primary or metastatic tumour, a tumour positive for BRAF oncogene expression, and a melanoma.

14. The pharmaceutical composition according to claim 13, wherein the tumour is a mammary tumour.

15. The medicament according to claim 2, wherein said phosphopeptide is conjugated with tumour targeting peptides and/or with antineoplastics.

16. A method to treat a medical condition of a patient, the method comprising
administering to the patient an effective amount of a phosphopeptide consisting of sequence RDGGGPGGDpYAAMGACPA (SEQ ID NO: 1), or a nucleotide sequence encoding for said phosphopeptide or a vector comprising said nucleotide sequence

17. A method to treat a tumour in a patient the method comprising
administering to the patient a phosphopeptide consisting of sequence RDGGGPGGDpYAAMGACPA (SEQ ID NO: 1), or a nucleotide sequence encoding for said phosphopeptide or a vector comprising said nucleotide sequence, in an effective amount to treat a tumour selected from the group consisting of a solid tumour, a primary or metastatic tumour, a HER2 and HER3 hyper-expressing and having elevated PI3K activity tumour or a primary or metastatic tumor, a tumour positive for BRAF oncogene expression, and a melanomas.

18. The method of claim 17, wherein the administering is performed in combination with one or more anti-tumour active principles.

19. The method of claim 17, wherein the wherein the phosphopeptide is conjugated with tumour targeting peptides and/or with antineoplastics.

20. The method of claim 17, wherein the tumour is a mammary tumour.


SEQUENCE LISTING (7 sequences)

SEQ ID NO: 1 - protein, 18 aa, Homo sapiens
  MOD_RES (10)..(10): PHOSPHORYLATION
RDGGGPGGDYAAMGACPA

SEQ ID NO: 2 - protein, 18 aa, artificial
  SEQ ID No1 scramble phosphopeptide
PYQMRYNADTDGERTTEP

SEQ ID NO: 3 - RNA, 19 nt, artificial
  RNA strand of a double strand oligonucleotide interfering with the expression of ErbB3
gcucuacgagaggugugag

SEQ ID NO: 4 - RNA, 19 nt, artificial
  RNA strand of a double strand oligonucleotide used as control in comparison to the double strand RNA oligonucleotide interfering with the expression of ErbB3
gcgcgcaacucuaccucua

SEQ ID NO: 5 - protein, 18 aa, Homo sapiens
  MOD_RES (11)..(11)
  MOD_RES (11)..(11): PHOSPHORYLATION
PTAGTTPDEDYEYMNRQR

SEQ ID NO: 6 - RNA, 19 nt, artificial
  RNA strand complementary to SEQ ID NO3
cucacaccucucguagagc

SEQ ID NO: 7 - RNA, 19 nt, artificial
  RNA strand complementary to SEQ ID NO4
uagagguagaguugcgcgc